(19) United States
(12) Patent Application Publication
(10) Pub. No.: US 2023/0233717 A1
(43) Pub. Date: Jul. 27, 2023

(54) SYSTEM AND METHOD FOR SUPPRESSING MICROBES HAVING A PHOTOSENSITIVE DEFENSE MECHANISM

(71) Applicant: KORRUS, INC., Los Angeles, CA (US)

(72) Inventor: Benjamin HARRISON, LOS ANGELES, CA (US)

(21) Appl. No.: 18/128,609

(22) Filed: Mar. 30, 2023

Related U.S. Application Data
(63) Continuation of application No. PCT/US2021/052946, filed on Sep. 30, 2021.
(60) Provisional application No. 63/085,817, filed on Sep. 30, 2020.

(51) Int. Cl. A61L 2/08; H05B 47/115; H05B 47/155; H05B 47/17

(52) U.S. Cl. CPC A61L 2/084; H05B 47/115; H05B 47/155; H05B 47/17; A61L 2202/11; A61L 2202/14

(57) ABSTRACT

A light system for suppressing a microbe having a photosensitive defense mechanism, said light system comprising a plurality of light sources comprising at least, a first light source configured for emitting a first light having a first wavelength suitable for photolyzing or otherwise inactivating the microbe; and a second light source configured for emitting a second light having a second wavelength, different from said first wavelength, suitable for disrupting said photosensitive defense mechanism; a controller for selectively powering said plurality of light sources in a plurality of modes to emit emitted light from said light system, said plurality of modes comprises at least a first mode and a second mode, wherein said emitted light is white light in at least one of said first mode or said second mode.

### CROSS-REFERENCE TO RELATED APPLICATION

This application claims the benefit of priority of U.S. Provisional Patent Application No. 63/085,817, filed Sep. 30, 2020; the entire disclosure of which is hereby incorporated herein by reference.

### FIELD OF INVENTION

The present application relates, generally, to antimicrobial light treatments, and, more specifically, to an antimicrobial light system for suppressing microbes having a photosensitive defense mechanism.

### BACKGROUND

Multidrug-resistant microbes have emerged as a significant problem in the healthcare field. For example, methicillin-resistant Staphylococcus aureus (MRSA) are particularly difficult to treat. Therefore, novel and nontraditional approaches are needed to suppress these microbes.

Antimicrobial light having a peak wavelength at 405-nm has been found recently to be a potential alternative treatment for suppressing microbes. The antimicrobial effects of this light are likely the result of the excitation of endogenous photosensitizing porphyrins and the subsequent generation of singlet oxygen, resulting in lipid peroxidation, DNA damage, cell wall damage, and cellular apoptosis of microbial cells. See LEON G. LEANSE ET AL. Dual-Wavelength Photo-Killing Of Methicillin-Resistant Staphylococcus Aureus, JCI Insight (Jun. 4, 2020) (https://doi.org/10.1172/jci.insight.134343) (Leanse et al.), hereby incorporated by reference.

Leanse et al. recognized that a particular phenotype of MRSA, golden colony phenotype, is more tolerant of 405 nm light than most other microbes. Golden colony MRSA has a membrane-bound carotenoid pigment comprising staphyloxanthin (STX), which is responsible for MRSA's characteristic golden color. Recent findings have demonstrated the antioxidant properties of STX. Accordingly, Leanse et al. theorized that the limited antimicrobial efficacy of 405 nm light was a direct result of STX because it is a known singlet oxygen scavenger.

Leanse et al. discovered that treatment of MRSA using 405-nm light could be improved by STX photolysis using preexposure with 460-nm light. In other words, the 460-nm light disrupted the antioxidant properties of the carotenoid pigment of MRSA such that the 405-nm light was able to excite the endogenous photosensitizing porphyrins of MRSA, causing generation of singlet oxygen, and hence subsequent lipid peroxidation, DNA damage, cell wall damage, and cellular apoptosis of MRSA cells.

Applicants recognize the need to take this discovery and expand its application while making the treatment commercially/aesthetically acceptable. The present invention fulfills this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

Applicant recognizes that Leanse et al.'s discovery of using two different wavelength lights—one wavelength to disrupt the microbes defense mechanism, and the other to generate singlet oxygen—is an important step forward in treating drug-resistant microbes. Applicant also recognizes that such an approach is not limited to MRSA, but also may apply to any microbe having a photosensitive defense mechanism. As used herein, the term “photosensitive defense mechanism” refers to any characteristic of a microbe that either inhibits antibacterial light from reaching the porphyrins of the microbe or scavenges the free radicals generated by the antibacterial light. For example, black mold, which is known to be toxic, has a pigment which functions as a “sunscreen” to prevent 405 nm light from reaching the porphyrins of the mold. Applicant recognizes that this sunscreen pigment can be photolyzed and the mold cells destroyed using a kill sequence of light having different wavelengths. In other words, by using a more nuanced approach to antibacterial light in which a sequence of different light having different wavelengths and different spectral power distributions (SPDs) is used not only to target the porphyrins of the microbe, but also to eliminate/disrupt/disable the photosensitive defense mechanism of the microbe, a more effective antibacterial light treatment for drug-resistant microbes can be realized.

Additionally, Applicant recognizes that any antibacterial light treatment of microbes in a residential or institutional environment where people or animals are present should use light of high quality, not far from the Planckian locus.

Accordingly, in one embodiment, the present invention relates to a lighting system for suppressing a microbe having a photosensitive defense mechanism. In one embodiment, the system comprises: (a) a plurality of light sources comprising at least, (i) a first light source configured for emitting first light configured to photolyze or otherwise inactivate the microbe; and (ii) a second light source configured for emitting a second light having a wavelength suitable for disrupting the photosensitive defense mechanism; (b) a controller for selectively powering the plurality of light sources in a plurality of modes to emit emitted light from the light system, the plurality of modes comprising at least a first mode and a second mode, wherein, in the first mode, at least the first light source is powered, and, in the second mode, at least the second light source is powered; and wherein the emitted light is white light in at least one of the first mode or the second mode, the white light having a chromaticity with Duv of less than 5 E-3 from the Planckian locus.

In another embodiment, the present invention relates to a method of using the light system described above. In one embodiment, the microbe is MRSA and the photosensitive defense mechanism is a pigment that absorbs free radicals released during the first mode, and wherein the second mode disrupts the pigment.

### BRIEF DESCRIPTION OF FIGURES

FIG. 1 shows one embodiment of the lighting system of the present invention.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

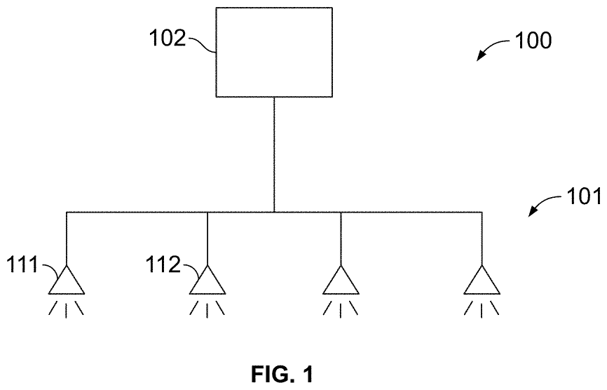

Referring to FIG. 1, one embodiment of a light system 100 of the present invention is shown. The light system 100 is configured for suppressing a microbe having a photosensitive defense mechanism. In this embodiment, the light system 100 comprises a plurality of light sources 101 comprising at least a first light source 111 configured for emitting first light configured to photolyze or otherwise inactivate the microbe; and a second light source 112 configured for emitting a second light having a wavelength suitable for disrupting the photosensitive defense mechanism. The light system 100 also comprises a controller 102 for selectively powering the plurality of light sources in a plurality of modes to emit emitted light from the light system, wherein the plurality of modes comprises at least a first mode and a second mode, in the first mode, at least the first light source is powered, and, in the second mode, at least the second light source is powered; and wherein the emitted light is white light in at least one of the first mode or the second mode, the white light having a chromaticity with Duv of less than 5 E-3 from the Planckian locus.

An important aspect of the present invention is the plurality of different light sources which are selectively powered by the controller to emit light from the system in different modes. Each mode corresponds to a different quality/property in the emitted light to achieve a different objective. For example, different modes may target a property of a microbe to suppress or eliminate the microbe. For example, in one mode, the light sources may be selectively powered to photolyze the microbes DNA/cell walls in one mode, and, in another mode, the light sources may be selectively powered disable/disrupt the microbe's photosensitive defense mechanism. In yet another mode, the light sources may be selectively powered to provide white light. Thus, the controller works with a “pallet” of light sources to emit different modes of light serving different purposes.

The different light sources should be configured to provide the controller with an array of spectrums sufficient to achieve the objective of the different modes. For example, in the as mentioned above, a first light source may be configured to photolyzed the microbe's DNA/cell wall or otherwise render the microbe inactive. In one embodiment, the first light source may be a ultraviolet (UV) light. In such an embodiment, the UV light may be UVa, UVb or UVc, and, in a particular embodiment, the UV light is UVa to minimize its harmful effects. In another embodiment, the first light source may emit light having a violet component. In such an embodiment, the first light source may have a peak wavelength between 380 nm and 420 nm, and, in a more particular embodiment, a peak wavelength of 380 nm, 395 nm or 405 nm, and, in a more particular embodiment, a peak wavelength of 405 nm. Additionally, in one embodiment, the first light source has a spectral power distribution (SPD) with an overall power between 380 nm and 780 nm, and a violet power fraction between 380 nm and 420 nm, wherein the violet power fraction is at least 25% of the overall power, and, in a more particular embodiment, at least 30% of the overall power, and, in a more particular embodiment, at least 35% of the overall power, and, in a more particular embodiment, at least 40% of the overall power.

In one embodiment, another light source is configured to disrupt the photosensitive defense mechanism of the microbe. Because different microbes have different defense mechanisms, the second light source will have different peak wavelengths and different SPDs depending on the microbe. One of skill in the art of antimicrobial light will be able to readily identify the wavelength and SPD of the light required to disrupt/disable/destroy a photosensitive defense mechanism of microbe. For example, in one embodiment, a light source is configured to disrupt staphyloxanthin (STX) in MRSA. As mentioned above, STX is photolyzed around 460 nm.

Accordingly, in one embodiment, the second light source has a peak wavelength of 450 nm and 500 nm, and, in a more particular embodiment, has a peak wavelength of 460 nm. In one embodiment, the second light source has an SPD having a blue power fraction between 420 nm and 480 nm which is at least 25% of the overall power between 380 nm and 420 nm, and, in a more particular embodiment, at least 30% of the overall power, and, in a more particular embodiment, at least 35% of the overall power, and, in a more particular embodiment, at least 40% of the overall power.

Although only a first and second light source are considered specifically in this disclosure, it should be understood that additional light sources are within the scope of the present invention. In other words, Applicant's disclosure is not limited to just two light sources, but may include additional light sources for achieving different modes as discussed below. For example, in one embodiment, a given microbe may have more than one photosensitive defense mechanism. In such a case, multiple light sources with peak wavelengths tuned to photolyzed the various photosensitive defense mechanisms may be required.

As mentioned above, the controller selectively powers the different light sources to emit emitted light of different modes. In other words, the controller can select various light sources of the plurality of light sources to deliver specific doses of light using the plurality of light sources available. In this way, in one embodiment, the controller can deliver a killer sequence of light modes to suppress/eliminate microbes. The dose of light in a mode can be determined by those of skill in the art without undue experimentation in light of this disclosure. In one embodiment, the dose of light depends on the power fractions of the SPD and the duration of the mode. For example, in one mode, the microbe is irradiated with light having an blue fraction of at least 25% of the overall power, and a peak wavelength of 460 nm for 10 minutes to disrupt the STX in MRSA.

The configuration of the different modes can vary according to application. For example, in one embodiment, at least the first light source is powered in the first mode, and at least the second light source is powered and the second mode. In a more particular embodiment, the second light source is not powered in the first mode, and the first and second light sources are powered in the second mode. In still another embodiment, the second light source is not powered in the first mode, and the first light source is not powered in the second mode. It should be obvious from this disclosure that as the number of light sources increases, the number of modes or permutations of light sources operating/not operating increases. For example, if there is a third light source for emitting a different light in one embodiment, then there may be a third mode in which just the third light source is powered, and a fourth mode in which the third light source and at least one of the first and second light sources is powered. Indeed, it should be understood that essentially any permutation of one or more of the light sources of the plurality of light sources operating within a given mode is possible.

As mentioned above, an important aspect of the present invention is having an antibacterial light treatment system that emits light of high quality. To this end, in one embodiment, one or more of the modes of the light system emits white light. As used herein, the term “white light” refers to light having a Duv within 0.005 of the Planckian locus. In a more particular embodiment, the white light has a Duv within 0.05 of the Planckian locus. In yet another embodiment, the white light has a Color Rendering Index (CRI) of at least 80, and, in a more particular embodiment, at least 85, and, in a more particular embodiment, at least 90.

In one embodiment, only one mode emits white light. In another embodiment, two or more modes emit white light. In yet another embodiment, all modes emit white light. It is generally preferred, although not necessary, that non-white modes of operation are minimized.

In light of this disclosure, those of skill in the art will understand how to balance light to produce white light. For example, in one embodiment, the light source itself is configured to emit white light (e.g. a light source with a large violet fraction may be balanced with a spectrum that is rich and cyan/green). Alternatively, different light sources may be mixed and matched in different modes such that the emitted light is white (e.g., a light source with a large violet fraction is mixed with a second light source having a large cyan/green fraction to form white light). Still other embodiments will be obvious to those of skill in the art in light of this disclosure.

For example, in one embodiment, in which the first mode comprises just the first light source and the second mode comprises both the first and second light sources, the first mode emits white light, but the addition of the second light source in the second mode results in emitted light which has a Duv greater than 0.005 from the Planckian locus. In an alternative embodiment, the first mode comprises only the first light source, and the second mode comprises only the second light source. In such a configuration, both modes may emit white light as defined herein.

In one particular embodiment, the controller is configured to selectively power the light sources to emit emitted light which is white light in one mode, and nonwhite light in another mode. For example, in one embodiment, the controller may be configured to emit light in a first mode and second mode. In the first mode, the first light source is powered to emit a peak wavelength of 405 nm to photolyze microbes, but which is otherwise balanced to emit white light. In the second mode, both the first light source and the second light source are powered. The second light source is powered to emit a peak wavelength of 460 nm light with a large blue SPD power fraction. Because the white light of the first light sources is combined with the blue light of the second light source, the emitted light of the second mode is not white light, but is shifted to the blue area of the gamut. In such a situation, it may not be preferable to have occupants in the irradiated space during the second mode as addressed below.

In one embodiment, the light system of the present invention comprises a sensor to detect occupancy of the space being irradiated by the emitted light. The purpose of this occupancy sensor is to determine when people or animals are in the irradiate space. Such information may be important in some embodiments in which one of more modes of the lighting system emit light which is not white light. In such a situation, the non-white light may be unpleasant to occupants within the space. Additionally, in embodiments in which the emitted light is UV (e.g., UVa), the light be harmful to the occupants. Accordingly, in one embodiment, the lighting system comprises a sensor to determine occupancy, and the controller only selectively powers the light sources to emit a nonwhite/UV light when the sensor determines that the space is unoccupied.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A light system for suppressing a microbe having a photosensitive defense mechanism, said light system comprising:
a plurality of light sources comprising at least,
a first light source configured for emitting a first light having a first wavelength suitable for photolyzing or otherwise inactivating the microbe; and
a second light source configured for emitting a second light having a second wavelength, different from said first wavelength, suitable for disrupting said photosensitive defense mechanism;
a controller for selectively powering said plurality of light sources in a plurality of modes to emit emitted light from said light system, said plurality of modes comprises at least a first mode and a second mode, wherein, in said first mode, at least said first light source is powered, and, in said second mode, at least said second light source is powered; and
wherein said emitted light is white light in at least one of said first mode or said second mode, said white light having a chromaticity with Duv of less than 5 E-3 from the Planckian locus.

2. The light system of claim 1, wherein said second light source is not powered in said first mode, and wherein said first and second light sources are powered in said second mode.

3. The light system of claim 2, wherein said emitted light is white light in said first mode.

4. The light system of claim 3, wherein said emitted light in said second mode has a chromaticity with a Duv of greater than 5 E-3 from the Planckian locus.

5. The light system of claim 4, further comprising a sensor for determining occupancy in a space being irradiated by said emitted light, and wherein said controller is configured to irradiate said space in said second mode when said space is not occupied.

6. The light system of claim 1, wherein said second light source is not powered in said first mode, and wherein said first light source is not powered in said second mode.

7. The light system of claim 6, wherein said emitted light is white light in both said first and second modes.

8. The light system of claim 1, wherein said controller alternates between said first and second modes.

9. The light system of claim 1, wherein said controller selectively powers said plurality of light sources in a kill sequence of said plurality of modes.

10. The light system of claim 1, wherein said controller selectively powers said plurality of light sources to emit light in at least one more additional mode.

11. The light system of claim 10, wherein said controller selectively powers said plurality of light sources in a kill sequence of said first mode, said second mode and said one or more additional modes.

12. The light system of claim 10, wherein each mode is characterized by a dose of a light having a particular spectral power distribution (SPD) different from the other modes.

13. The light system of claim 1, wherein said first light has a peak wavelength between 380 nm and 420 nm, and wherein the first light source has an SPD with an overall power between 380 nm and 780 nm, and a violet power fraction between 380 nm and 420 nm, wherein the violet power fraction is at least 25% of the overall power

14. The light system of claim 13, wherein said first light has a peak wavelength of 380 nm, 395 nm or 405 nm.

15. The light system of claim 14, wherein said first light has a peak wavelength of 405 nm

16. The light system of claim 14, wherein said second light has an SPD with an overall power between 380 nm and 780 nm, and a blue power fraction between 450 and 500 nm, wherein said blue power fraction is at least 25% of said overall power.

17. The light system of claim 16, wherein said second light has a peak wavelength of 450 nm and 500 nm.

18. The light system of claim 17, wherein said second light has a peak wavelength of 460 nm.

19. A method of using the light system of claim 1.

20. A method of using the light system of claim 18, wherein said microbe is MRSA and said photosensitive defense mechanism is a pigment that absorbs free radicals released during said first mode, and wherein said second mode disrupts said pigment.
